(19) United States
(12) Patent Application Publication
(10) Pub. No.: US 2005/0249681 A1
(43) Pub. Date: Nov. 10, 2005

(54) Use of diketopiperazine derivatives as photostable uv-filters in cosmetic and pharmaceutical preparations

(76) Inventors: Thomas Heidenfelder, Dannstadt (DE); Kristin Tiefensee, Bad Duerkheim (DE); Thomas Wunsch, Speyer (DE); Robert Parker, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/523,794

(22) PCT Filed: Jul. 24, 2003
(86) PCT No.: PCT/EP03/08109

§ 371 Date: Feb. 9, 2005

(30) Foreign Application Priority Data
Aug. 15, 2002 (DE) 102-38-144.5

(51) Int. Cl. A61K 7/42; A61K 7/06

(52) U.S. Cl. 424/59; 424/70.9

(57) ABSTRACT

Use of diketopiperazine derivatives of the formula I, 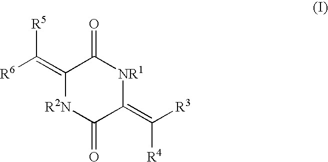 in which the variables have the meanings explained in the description, as photostable UV filters in cosmetic and pharmaceutical preparations for protecting the human skin or human hair against solar rays, alone or together with compounds which absorb in the UV region known per se for cosmetic and pharmaceutical preparations.

The invention relates to the use of diketopiperazine derivatives as photostable UV filters in cosmetic and pharmaceutical preparations for protecting the human epidermis or human hair against UV radiation, especially in the range from 320 to 400 nm.

The sunscreens employed in cosmetic and pharmaceutical preparations have the task of preventing, or at least diminishing the consequences of, harmful effects of sunlight on the human skin. However, these sunscreens also serve to protect other ingredients from decomposition or degradation by UV radiation. In hair cosmetic formulations the aim is to reduce damage to the keratin fibers by UV rays.

The sunlight reaching the surface of the earth contains proportions of UV-B radiation (280 to 320 nm) and UV-A radiation (>320 nm), which are directly adjacent to the visible light region. The effect on the human skin is manifested, particularly in the case of UV-B radiation, by sunburn. Accordingly, the industry offers a relatively large number of substances which absorb UV-B radiation and thus prevent sunburn.

Dermatological investigations have now shown that UV-A radiation is also perfectly capable of causing skin damage and allergies by, for example, damaging the keratin or elastin. This reduces the elasticity and water storage capacity of the skin, i.e. the skin becomes less supple and tends to form wrinkles. The noticeably high incidence of skin cancer in regions where the sun's radiation is strong shows that damage to the genetic information in the cells is evidently also caused by sunlight, specifically by UV-A radiation. All these findings would therefore suggest the need to develop efficient filter substances for the UV-A region.

There is a growing demand for sunscreens for cosmetic and pharmaceutical preparations which can be used in particular as UV-A filters and whose absorption maxima ought therefore to be in the range from about 320 to 380 nm. In order to achieve the required effect using the minimum amount, sunscreens of this type ought additionally to have a high specific absorbance. Sunscreens for cosmetic products must also meet a large number of other requirements, for example good solubility in cosmetic oils, high stability of the emulsions produced with them, toxicological acceptability, and low intrinsic odor and low intrinsic color.

Another requirement which sunscreens must meet is adequate photostability. However, this is only inadequately ensured, if at all, with the UV-A-absorbing sunscreens hitherto available.

French Patent No. 2 440 933 describes 4-(1,1-dimethylethyl)-4′-methoxydibenzoylmethane as a UV-A filter. It is proposed to combine this specific UV-A filter, which is sold by GIVAUDAN under the name “PARSOL 1789”, with various UV-B filters in order to absorb all UV rays having a wavelength from 280 to 380 nm.

However, this UV-A filter does not have sufficient photochemical stability, when used alone or in combination with UV-B filters, to ensure sustained protection of the skin during sunbathing for prolonged periods, which means that repeated applications at regular and short intervals are required if effective protection of the skin from all UV rays is desired.

For this reason, EP-A-0 514 491 discloses the stabilization of the insufficiently photostable UV-A filters by adding 2-cyano-3,3-diphenylacrylic esters which themselves act as filters in the UV-B region.

It has furthermore already been proposed in EP-A-0 251 398 and EP-A-0 416 837 to combine chromophores absorbing UV-A radiation and UV-B radiation into one molecule using a linker. This has the disadvantage that firstly a free combination of UV-A and UV-B filters in the cosmetic preparation is no longer possible, and that difficulties in the chemical linkage of the chromophores allow only certain combinations.

It is an object of the present invention to propose sunscreens for cosmetic and pharmaceutical purposes which absorb in the UV-A region with high absorbance, which are photostable, have low intrinsic color, i.e. a sharp band structure, and are soluble in oil or water depending on the substituent.

We have found that this object is achieved according to the invention by the use of diketopiperazines of the formula I, 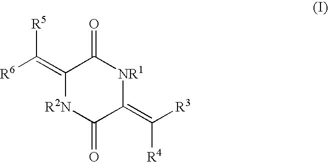
where
R¹ and R² may in each case, independently of one another, be identical or different and are hydrogen or C₁-C₁₂-alkyl and R³, R⁴, R⁵, R⁶ may in each case, independently of one another, be identical or different and are hydrogen, C₁-C₁₂-alkyl or an aryl radical,
with the proviso that at least one radical should be aryl,
as photostable UV filters in cosmetic and pharmaceutical preparations for protecting the human skin or human hair against solar rays, alone or together with compounds which absorb in the UV region known for cosmetic and pharmaceutical preparations.

Preference is given to using those compounds in which the group R³/R⁴ is identical to the group R⁵/R⁶ and R¹ and R² are hydrogen.

C₁-C₁₂-Alkyl radicals which may be mentioned are branched or unbranched C₁-C₂₀-alkyl chains, preferably methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, l-ethyl-l-methylpropyl, 1-ethyl-2-methylpropyl, n-heptyl, n-octyl, 2-ethylhexyl, n-nonyl, n-decyl, n-undecyl or n-dodecyl.

Aryl is understood as meaning aromatic rings or ring systems having 6 to 18 carbon atoms in the ring system, for example benzyl, phenyl or naphthyl, which may optionally be substituted with one or more radicals, such as halogen, e.g. fluorine, chlorine or bromine, cyano, nitro, amino, C₁-C₁₂-alkylamino, C₁-C₁₂-dialkylamino, hydroxyl, C₁-C₄-alkyl, C₁-C₄-alkoxy or other radicals. Preference is given to optionally substituted phenyl, methoxyphenyl, cyanophenyl and naphthyl.

Suitable alkoxy radicals are those with 1 to 4 carbon atoms.

Examples which can be mentioned are:

| | |
|---|---|
| methoxy | ethoxy |
| isopropoxy | n-propoxy |
| 1-methylpropoxy | n-butoxy |
| 3-methylbutoxy | 2-methylpropoxy |
| 2,2-dimethylpropoxy | 1,1-dimethylpropoxy |
| 1-methyl-1-ethylpropoxy | |

Suitable mono- or dialkylamino radicals are those which contain alkyl radicals having 1 to 12 carbon atoms, such as, for example, methyl, n-propyl, n-butyl, 2-methylpropyl, 1,1-dimethylpropyl, hexyl, heptyl, 2-ethylhexyl, isopropyl, 1-methylpropyl, n-pentyl, 3-methylbutyl, 2,2-dimethylpropyl, 1-methyl-1-ethylpropyl and octyl.

Examples of compounds of the formula I may be the compounds listed below:
(3Z,6Z)-6-benzylidene-3-(4-methoxybenzylidene)-2,5-piperazinedione,
(3Z,6Z)-6-benzylidene-3-(2,6-dichlorobenzylidene)-2,5-piperazinedione,
(3Z,6Z)-3-(4-acetoxybenzylidene)-6-benzylidene-2,5-piperazinedione,
(3Z,6Z)-6-benzylidene-3-(4-nitrobenzylidene)-2,5-piperazinedione, 3,6-dibenzylidene-2,5-piperazinedione,
(3Z,6Z)-6-benzylidene-3-(3-nitrobenzylidene)-2,5-piperazinedione,
(3Z,6Z)-6-benzylidene-3-(2-nitrobenzylidene)-2,5-piperazinedione,
(3Z,6Z)-6-benzylidene-3-(4-ethoxybenzylidene)-2,5-piperazinedione,
(3Z,6Z)-6-benzylidene-3-(4-cyanobenzylidene)-2,5-piperazinedione,
(3Z,6Z)-3-(4-aminobenzylidene)-6-benzylidene-2,5-piperazinedione,
(3Z,6Z)-3-(3-acetoxybenzylidene)-6-benzylidene-2,5-piperazinedione,
(3Z,6Z)-3-(2-acetoxybenzylidene)-6-benzylidene-2,5-piperazinedione,
(3Z,6Z)-6-benzylidene-3-(3-hydroxybenzylidene)-2,5-piperazinedione,
(3Z,6Z)-3-(4-acetamidobenzylidene)-6-benzylidene-2,5-piperazinedione,
(3Z,6Z)-3-(2-acetamidobenzylidene)-6-benzylidene-2,5-piperazinedione,
(3Z,6Z)-3-(2-aminobenzylidene)-6-benzylidene-2,5-piperazinedione,
(3Z,6Z)-3-(4-acetoxymethylbenzylidene)-6-benzylidene-2,5-piperazinedione,
(3Z,6Z)-3-(4-acetamidomethylbenzylidene)-6-benzylidene-2,5-piperazinedione,
(3Z,6Z)-3,6-dibenzylidene-2,5-piperazinedione,
(3Z,6Z)-6-benzylidene-3-(4-butoxybenzylidene)-2,5-piperazinedione,
(3Z,6Z)-6-benzylidene-3-(4-tert-butylbenzylidene)-2,5-piperazinedione,
(3Z,6Z)-6-benzylidene-3-(4-isopropoxybenzylidene)-2,5-piperazinedione,
(3Z,6Z)-6-benzylidene-3-(2,4-difluorobenzylidene)-2,5-piperazinedione,
(3Z,6Z)-6-benzylidene-3-(2-bromobenzylidene)-2,5-piperazinedione,
(3Z,6Z)-6-benzylidene-3-(4-methylthiomethylbenzylidene)-2,5-piperazinedione,
(3Z,6Z)-6-benzylidene-3-(3-thioacetoxymethylbenzylidene)-2,5-piperazinedione,
methyl 3-((3Z,6Z)-6-benzylidene-2,5-dioxopiperazine-3-ylidene)methylbenzoate,
(3Z,6Z)-6-benzylidene-3-(3-mercaptomethylbenzylidene)-2,5-piperazinedione,
(3Z,6Z)-6-benzylidene-3-(4-tert-butoxycarbonylaminobenzylidene)-2,5-piperazinedione,
(3Z,6Z)-6-benzylidene-3-(4-(3-N,N-dimethylaminopropoxy)-benzylidene)-2,5-piperazinedione,
(3Z,6Z)-6-benzylidene-3-(4-thioacetoxymethylbenzylidene)-2,5-piperazinedione,
(3Z,6Z)-6-benzylidene-3-(2-chloro-4-hydroxybenzylidene)-2,5-piperazinedione,
(3Z,6Z)-6-benzylidene-3-(3,4-dimethoxybenzylidene)-2,5-piperazinedione,
methyl 4-[(3Z,6Z)-6-benzylidene-2,5-dioxopiperazine-3-ylidene)methylphenoxyacetate,
methyl 4-(4-[(3Z,6Z)-6-benzylidene-2,5-dioxopiperazine-3-ylidene)methylbenzylcarbamoyl)butanoate,
methyl 4-(4-[(3Z,6Z)-6-benzylidene-2,5-dioxopiperazine-3-ylidene)methylbenzylcarbamoyl)pentanoate,
methyl 5-[4-[(3Z,6Z)-6-benzylidene-2,5-dioxopiperazine-3-ylidene)methylphenoxy]pentanoate,
5-[4-[(3Z,6Z)-6-benzylidene-2,5-dioxopiperazine-3-ylidene)methylphenoxy]pentanoic acid,
(3Z,6Z)-6-benzylidene-3-(4-(2-N,N-dimethylaminoethoxy)benzylidene)-2,5-piperazinedione hydrochloride,
(3Z,6Z)-6-benzylidene-3-(4-(2-N,N-dimethylaminoethoxy)benzylidene)-2,5-piperazinedione,
4-[(3Z,6Z)-6-benzylidene-2,5-dioxopiperazine-3-ylidene)methylphenoxyacetic acid,
(3Z,6Z)-3-(4-acetamidobenzylidene)-6-methoxybenzylidene)-2,5-piperazinedione,
(3Z,6Z)-6-(4-methoxybenzylidene)-3-(2-nitrobenzylidene)-2,5-piperazinedione,
(3Z,6Z)-3-(2,6-dichlorobenzylidene)-6-(4-methoxybenzylidene)-2,5-piperazinedione,
(3Z,6Z)-3-(4-hydroxybenzylidene)-6-(4-methoxybenzylidene)-2,5-piperazinedione,
(3Z,6Z)-3-(4-acetoxybenzylidene)-6-(4-methoxybenzylidene)-2,5-piperazinedione,
(3Z,6Z)-3-(4-methoxybenzylidene)-6-(4-N-methylacetamidbenzylidene)-2,5-piperazinedione,
(3Z,6Z)-3-(4-methoxybenzylidene)-6-(4-methylsulfonylbenzylidene)-2,5-piperazinedione,
(3Z,6Z)-3-(4-butoxybenzylidene)-6-(4-methoxybenzylidene)-2,5-piperazinedione,
(3Z,6Z)-3-(4-isopropoxybenzylidene)-6-(4-methoxybenzylidene)-2,5-piperazinedione,
(3Z,6Z)-3-(4-methoxybenzylidene)-6-(4-tert-butylbenzylidene)-2,5-piperazinedione,
(3Z,6Z)-3-(2-bromobenzylidene)-6-(4-methoxybenzylidene)-2,5-piperazinedione,
(3Z,6Z)-(4-methoxybenzylidene)-6-(4-tert-butoxycarbonylaminomethylbenzylidene)-2,5-piperazinedione,
(3Z,6Z)-3-(4-methoxybenzylidene)-6-(4-methylthiomethylbenzylidene)-2,5-piperazinedione,
(3Z,6Z)-3-(4-methoxybenzylidene)-6-(4-methylsulfonylmethylbenzylidene)-2,5-piperazinedione,
(3Z,6Z)-3-(4-methoxybenzylidene)-6-(3-thioacetoxymethylbenzylidene)-2,5-piperazinedione,
(3Z,6Z)-3-(4-aminomethylbenzylidene)-6-(4-methoxybenzylidene)-2,5-piperazinedione,
(3Z,6Z)-3-(2,4-difluorobenzylidene)-6-(4-methoxybenzylidene)-2,5-piperazinedione,
(3Z,6Z)-3-(4-methoxybenzylidene)-6-(2-trifluoromethylbenzylidene)-2,5-piperazinedione,
(3Z,6Z)-3-(2,4-dimethoxybenzylidene)-6-(4-methoxybenzylidene)-2,5-piperazinedione,
4-[(3Z,6Z)-6-(4-methoxybenzylidene)-2,5-dioxopiperazine-3-ylide-ne)methylbenzamide,
(3Z,6Z)-3-(4-methoxybenzylidene)-6-(4-trimethylacetoxybenzylidene)-2,5-piperazinedione,
(3Z,6Z)-3-(4-methoxybenzylidene)-6-(4-methoxycarbonylbenzylidene)-2,5-piperazinedione,
(3Z,6Z)-3-(2-chloro-4-hydroxybenzylidene)-6-(4-methoxybenzylidene)-2,5-piperazinedione,
(3Z,6Z)-3-(4-acetoxyacetylaminobenzylidene)-6-(4-methoxybenzylidene)-2,5-piperazinedione,
(3Z,6Z)-3-(3,4-dimethoxybenzylidene)-6-(4-methoxybenzylidene)-2,5-piperazinedione, methyl
4-[(3Z,6Z)-6-(4-methoxybenzylidene)-2,5-dioxopiperazine-3-ylide-ne)methylbenzylcarbamoyl)butanoate,
(3Z,6Z)-3-(4-methoxybenzylidene)-6-(2-naphthylmethylene)-2,5-piperazinedione,
(3Z,6Z)-3-(4-hydroxyacetylaminobenzylidene)-6-(4-methoxybenzylidene)-2,5-piperazinedione,
(3Z,6Z)-3-(4-acetamidobenzylidene)-6-benzylidene-2,5-piperazinedione
(3Z,6Z)-3,6-di(3-nitrobenzylidene)-2,5-piperazinedione,
(3Z,6Z)-3-(4-acetamidobenzylidene)-6-(2,6-dichlorobenzylidene)-2,5-piperazinedione,
(3Z,6Z)-3-(4-acetamidobenzylidene)-6-(4-chlorobenzylidene)-2,5-piperazinedione,
(3Z,6Z)-3-(4-acetamidobenzylidene)-6-(4-acetoxymethylbenzylidene)-2,5-piperazinedione,
(3Z,6Z)-3-(4-acetamidobenzylidene)-6-(2-fluorobenzylidene)-2,5-piperazinedione,
(3Z,6Z)-3-(4-acetamidobenzylidene)-6-(4-fluoromethylbenzylidene)-2,5-piperazinedione,
(3Z,6Z)-6-(benzylidene)-3-(2,4-difluorobenzylidene)-2,5-piperazinedione,
(3Z,6Z)-6-(4-acetamidobenzylidene)-3-(2-trifluoromethylbenzylidene)-2,5-piperazinedione,
(3Z,6Z)-6-(4-acetamidobenzylidene)-3-(2-bromobenzylidene)-2,5-piperazinedione,
(3Z,6Z)-3-(4-acetamidobenzylidene)-6-(4-trimethylacetoxybenzylidene)-2,5-piperazinedione,
(3Z,6Z)-3-(4-acetamidobenzylidene)-6-(4-dimethylaminobenzylidene)-2,5-piperazinedione,
(3Z,6Z)-3-(4-acetamidobenzylidene)-6-(4-tert-butoxycarbonylaminomethylbenzylidene)-2,5-piperazinedione.

The compounds of the formula I to be used according to the invention are known from the prior art (DE 39 18 178) and can, for example, be prepared in accordance with the synthesis routes detailed in EP 655 060 or as in J. Prakt. Chemie 1966, 32, 158-166 or J. Heterocyclic. Chem. 1988, 25, 591-597.

The present invention further provides cosmetic and pharmaceutical preparations which comprise 0.1 to 10% by weight, preferably 1 to 7% by weight, based on the total amount of the cosmetic and pharmaceutical preparation, of one or more of the compounds of the formula I together with compounds which absorb in the UV-A and UV-B region known per se for cosmetic and pharmaceutical preparations as light protection agents, where the compounds of the formula I are generally used in a lesser amount than the UV-B-absorbing compounds.

The cosmetic and pharmaceutical preparations which contain light protection agents are generally based on a carrier which comprises at least one oil phase. However, preparations based solely on water are also possible if compounds having hydrophilic substituents are used. Accordingly, oils, oil-in-water and water-in-oil emulsions, creams and pastes, lip protection stick compositions or non-greasy gels are suitable.

Suitable emulsions are, inter alia, also O/W macroemulsions, O/W microemulsions or O/W/O emulsions containing diketopiperazines of the formula I in dispersed form, the emulsions being obtainable by phase inversion technology, as in DE-A-197 26 121.

Conventional cosmetic auxiliaries which may be suitable as additives are, for example, coemulsifiers, fats and waxes, stabilizers, thickeners, biogenic active substances, film formers, fragrances, dyes, pearlizing agents, preservatives, pigments, electrolytes (e.g. magnesium sulfate) and pH regulators. Suitable and preferred coemulsifiers are known W/O and also O/W emulsifiers such as polyglycerol esters, sorbitan esters or partially esterified glycerides. Typical examples of fats are glycerides; waxes which may be mentioned are, inter alia, beeswax, paraffin wax or microwaxes, possibly combined with hydrophilic waxes. Stabilizers which can be employed are metal salts of fatty acids such as, for example, magnesium stearate, aluminum stearate and/or zinc stearate. Examples of suitable thickeners are crosslinked polyacrylic acids and derivatives thereof, polysaccharides, in particular xanthan gum, guar guar, agar agar, alginates and tyloses, carboxymethylcellulose and hydroxyethylcellulose, also fatty alcohols, monoglycerides and fatty acids, polyacrylates, polyvinyl alcohol and polyvinylpyrrolidone. Examples of biogenic active ingredients are plant extracts, protein hydrolysates and vitamin complexes. Examples of customary film formers are hydrocolloids such as chitosan, microcrystalline chitosan or quaternized chitosan, polyvinylpyrrolidone, vinylpyrrolidone/vinyl acetate copolymers, polymers of the acrylic acid series, quaternary cellulose derivatives and similar compounds. Examples of suitable preservatives are formaldehyde solution, p-hydroxybenzoate or sorbic acid. Examples of suitable pearlizing agents are glycol distearic esters such as ethylene glycol distearate, but also fatty acids and fatty acid monoglycol esters. Dyes which can be used are the substances suitable and approved for cosmetic purposes, as listed, for example, in the publication “Kosmetische Färbemittel” [Cosmetic Colorants] from the Farbstoffkommission der Deutschen Forschungsgemeinschaft [Dyes Commission of the German Research Society], published by Verlag Chemie, Weinheim, 1984. These dyes are normally used in a concentration of from 0.001 to 0.1% by weight, based on the total mixture.

An additional content of antioxidants is generally preferred.

Thus, it is possible to use as favorable antioxidants all antioxidants which are customary or suitable for cosmetic and/or dermatological applications.

The antioxidants are advantageously chosen from the group 20 consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl, and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. ascorbyl palmitate, Mg ascorbylphosphate, ascorbylacetate), tocopherol and derivatives (e.g. vitamin E acetate, tocotrienol), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned antioxidants (one or more compounds) in the preparations is preferably from 0.001 to 30% by weight, particularly preferably from 0.05 to 20% by weight, in particular from 1 to 10% by weight, based on the total weight of the preparation.

If vitamin E and/or derivatives thereof are used as the antioxidant(s), it is advantageous to choose their particular concentration from the range 0.001 to 10% by weight, based on the total weight of the formulation.

If vitamin A and/or derivatives thereof, or carotenoids are the antioxidant(s), it is advantageous to choose their particular concentration from the range 0.001 to 10% by weight, based on the total weight of the formulation.

Customary oil components in cosmetics are, for example, paraffin oil, glyceryl stearate, isopropyl myristate, diisopropyl adipate, cetylstearyl 2-ethylhexanoate, hydrogenated polyisobutene, vaseline, caprylic/capric triglycerides, microcrystalline wax, lanolin and stearic acid.

The total amount of auxiliaries and additives can be from 1 to 80% by weight, preferably from 6 to 40% by weight, and the nonaqueous fraction (“active substance”) can be from 20 to 80% by weight, preferably from 30 to 70% by weight, based on the compositions. The compositions can be prepared in a manner known per se, i.e. for example by hot, cold, hot-hot/cold or PIT emulsification. This is a purely mechanical process; no chemical reaction takes place.

Such sunscreen preparations can accordingly be in liquid, paste or solid form, for example as water-in-oil creams, oil-in-water creams and lotions, aerosol foam creams, gels, oils, marking pencils, powders, sprays or alcoholic-aqueous lotions.

Finally, it is also possible to co-use further substances which absorb in the UV region and are known per se provided they are stable in the overall system of the combination of UV filters to be used according to the invention.

The greatest part of the light protection agents in the cosmetic and pharmaceutical preparations used to protect the human epidermis consists of compounds which absorb UV light in the UV-B region i.e. in the range from 280 to 320 nm. For example, the fraction of UV-A absorbers to be used according to the invention is 10 to 90% by weight, preferably 20 to 50% by weight, based on the total amount of substances which absorb UV-B and UV-A.

Suitable UV filter substances which are used in combination with the compounds of the formula I to be used according to the invention are any UV-A and UV-B filter substances. Examples which may be mentined are:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one | 52793-97-2 |
| | methylsulfate | |
| 3 | 3,3,5-Trimethylcyclohexylsalicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its | 27503-81-7 |
| | potassium, sodium and triethanolamine salts | |
| 6 | 3,3′-(1,4-Phenylenedimethine)-bis(7,7-dimethyl- | 90457-82-2 |
| | 2-oxobicyclo[2.2.1]heptane-1-methanesulfonic | |
| | acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and its sodium salt | |
| 13 | 3-(4′-Sulfo)benzylidenebornan-2-one and salts | 58030-58-6 |
| 14 | 3-Benzylidenebornan-2-one | 16087-24-8 |
| 15 | 1-(4′-Isopropylphenyl)-3-phenylpropane-1,3- | 63260-25-9 |
| | dione | |
| 16 | 4-Isopropylbenzylsalicylate | 94134-93-7 |
| 17 | 2,4,6-Trianiline(o-carbo-2′-ethylhexyl-1′-oxy)- | 88122-99-0 |
| | 1,3,5-triazine | |
| 18 | 3-Imidazol-4-yl-acrylic acid and its ethyl | 104-98-3 |
| | ester | |
| 19 | Ethyl 2-cyano-3,3-diphenylacrylate | 5232-99-5 |
| 20 | 2′-Ethylhexyl 2-cyano-3,3-diphenylacrylate | 6197-30-4 |
| 21 | Menthyl o-aminobenzoate or: | 134-09-8 |
| | 5-methyl-2-(1-methylethyl)-2-aminobenzoate | |
| 22 | Glyceryl p-aminobenzoate or: | 136-44-7 |
| | 1-glyceryl 4-aminobenzoate | |
| 23 | 2,2′-Dihydroxy-4-methoxybenzophenone | 131-53-3 |
| | (dioxybenzone) | |
| 24 | 2-Hydroxy-4-methoxy-4-methylbenzophenone | 1641-17-4 |
| | (mexonone) | |
| 25 | Triethanolamine salicylate | 2174-16-5 |
| 26 | Dimethoxyphenylglyoxalic acid or: | 4732-70-1 |
| | sodium 3,4-dimethoxyphenylglyoxalate | |
| 27 | 3-(4′-Sulfo)benzylidenebornan-2-one and its | 56039-58-8 |
| | salts | |
| 28 | 4-tert-Butyl-4\grave{ }-methoxy-dibenzoylmethane | 70356-09-1 |
| 29 | 2,2\grave{ },4,4\grave{ }-Tetrahydroxybenzophenone | 131-55-5 |
| 30 | 2,2\grave{ }-Methylenebis[6(2H-benzotriazol-2-yl)- | 103597-45-1 |
| | 4-(1,1,3,3-tetramethylbutyl)phenol] | |
| 31 | 2,2\grave{ }-(1,4-Phenylene)bis-1H-benzimidazole-4,6-di | 180898-37-7 |
| | sulfonic acid, Na salt | |
| 32 | 2,4-bis[4-(2-Ethylhexyloxy)-2-hydroxy]phenyl- | 187393-00-6 |
| | 6-(4-methoxyphenyl)-(1,3,5)-triazine | |

In addition, the cosmetic and dermatological preparations according to the invention can advantageously comprise further inorganic pigments based on metal oxides and/or other metal compounds which are insoluble or virtually insoluble in water, in particular the oxides of titanium (TiO₂), Zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and admixtures of such oxides. The pigments are particularly preferably based on TiO₂ and ZnO. For the purposes of the present invention, it is particularly advantageous, but not obligatory, for the inorganic pigments to be present in hydrophobic form, i.e. to have been surface-treated to repel water. This surface treatment can involve providing the pigments with a thin hydrophobic layer in a manner known per se, as described in DE-A-33 14 742.

To protect human hair against UV rays, the light protection agents of the formula I according to the invention can be incorporated into shampoos, lotions, gels, hairsprays, aerosol foam creams or emulsions in concentrations of from 0.1 to 10% by weight, preferably from 1 to 7% by weight. The respective formulations can be used, inter alia, for washing, coloring and for styling the hair.

The compounds to be used according to the invention are generally characterized by a particularly high absorption capacity in the UV-A radiation region with a sharp band structure. In addition, they are readily soluble in cosmetic oils and can be readily incorporated into cosmetic formulations. The emulsions prepared using the compounds I are characterized in particular by their high stability, the compounds I themselves by their high photostability, and the preparations prepared with I by their pleasant feel on the skin.

Thie UV filter action of the compounds of the formula I according to the invention can also be utilized for stabilizing active ingredients and auxiliaries in cosmetic and pharmaceutical formulations.

The examples below illustrate the preparation and use of the diketopiperazines.

### EXAMPLES

### Example 1

### Synthesis of the diketopiperazines

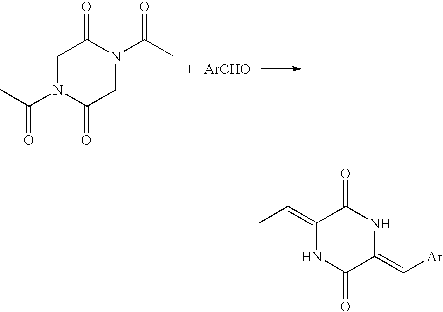

The compounds a-c were prepared analogously to the synthesis described in EP 65 5 060. The piperazines were obtained from the reaction of 1,4-diacetyl-2,5-piperazinedione with corresponding benzaldehydes in the presence of triethylamine.

TABLE 1

| | Ar | λₘₐₓ | E¹₁ |
|---|---|---|---|
| a | Ph | 332 | 1106 |
| b | p-CN-C₆H₄ | 354 | 554 |
| c | p-OMe-C₆H₄ | 334 | 736 |

### Example 2

### Standardized Method to Determine Photostability (Sun Test)

A 5% by weight alcoholic solution of the sunscreen to be tested is applied, using an Eppendorf pipette (20 μl), to the milled area on a small glass plate. Owing to the presence of the alcohol, the solution distributes uniformly on the roughened glass surface. The amount applied corresponds to the amount of sunscreen required to obtain an average sun protection factor in sun creams. In the test, 4 small glass plates are irradiated in each case. The evaporation time and the irradiation each last for 30 minutes. The glass plates are cooled slightly during the irradiation by a water cooling system located at the base of the sun test apparatus. The temperature inside the sun test apparatus during the irradiation is 40° C. After the samples have been irradiated, they are washed with ethanol into a dark 50 ml volumetric flask and measured using a photometer. The blank samples are applied in the same way to small glass plates and evaporated at room temperature for 30 minutes. Like the other samples, they are washed off with ethanol and diluted to 100 ml and measured.

General procedure for preparing emulsions for cosmetic purposes

All of the oil-soluble constituents are heated to 85° C. in a stirred vessel. When all of the constituents are molten or are present as liquid phase, the aqueous phase is incorporated with homogenization. The emulsion is cooled to about 40° C. with stirring, is perfumed and homogenized, and is then cooled to 25° C. with continuous stirring.

Preparations

### Example 3

### Lip Care Composition

Mass Content

(% by wt.)

| ad 100 | Eucerinum anhydricum |
|---|---|
| 10.00 | glycerol |
| 10.00 | titanium dioxide, micronized |
| 5.00 | compound (a) in Table 1 |
| 8.00 | octyl methoxycinnamate |
| 5.00 | zinc oxide |
| 4.00 | castor oil |
| 4.00 | pentaerythrithyl stearate/caprate/caprylate adipate |
| 3.00 | glyceryl stearate SE |
| 2.00 | beeswax |
| 2.00 | microcrystalline wax |
| 2.00 | quaternium-18 bentonite |
| 1.50 | PEG-45/dodecyl glycol copolymer |

### Example 4

### Lip Care Composition

Mass content

(% by wt.)

| ad 100 | Eucerinum anhydricum |
|---|---|
| 10.00 | glycerol |
| 10.00 | titanium dioxide, micronized |
| 5.00 | compound (b) in Table 1 |
| 8.00 | octyl methoxycinnamate |
| 5.00 | zinc oxide |
| 4.00 | castor oil |
| 4.00 | pentaerythrithyl stearate/caprate/caprylate adipate |
| 3.00 | glyceryl stearate SE |
| 2.00 | beeswax |
| 2.00 | microcrystalline wax |
| 2.00 | quaternium-18 bentonite |
| 1.50 | PEG-45/dodecyl glycol copolymer |

### Example 5

### Sunblock Composition Containing Micropigments

Mass content

(% by wt.)

| ad 100 | water |
|---|---|
| 10.00 | octyl methoxycinnamate |
| 6.00 | PEG-7 hydrogenated castor oil |
| 6.00 | titanium dioxide, micronized |
| 5.00 | compound (a) in Table 1 |
| 5.00 | mineral oil |
| 5.00 | isoamyl p-methoxycinnamate |
| 5.00 | propylene glycol |
| 3.00 | jojoba oil |
| 3.00 | 4-methylbenzylidenecamphor |
| 2.00 | PEG-45/dodecyl glycol copolymer |
| 1.00 | dimethicone |
| 0.50 | PEG-40 hydrogenated castor oil |
| 0.50 | tocopheryl acetate |
| 0.50 | phenoxyethanol |
| 0.20 | EDTA |

### Example 6

### Sunblock Composition Containing Micropigments

Mass content

(% by wt.)

| ad 100 | water |
|---|---|
| 10.00 | octyl methoxycinnamate |
| 6.00 | PEG-7 hydrogenated castor oil |
| 6.00 | titanium dioxide, micronized |
| 5.00 | compound (a) in Table 1 |
| 5.00 | mineral oil |
| 5.00 | isoamyl p-methoxycinnamate |
| 5.00 | propylene glycol |
| 3.00 | jojoba oil |
| 3.00 | 4-methylbenzylidenecamphor |
| 2.00 | PEG-45/dodecyl glycol copolymer |
| 1.00 | dimethicone |
| 0.50 | PEG-40 hydrogenated castor oil |
| 0.50 | tocopheryl acetate |
| 0.50 | phenoxyethanol |
| 0.20 | EDTA |

### Example 7

### Non-Greasy Gel

Mass content

(% by wt.)

| ad 100 | water |
|---|---|
| 8.00 | octyl methoxycinnamate |
| 7.00 | titanium dioxide, micronized |
| 5.00 | compound (b) in Table 1 |
| 5.00 | glycerol |
| 5.00 | PEG-25 PABA |
| 1.00 | 4-methylbenzylidenecamphor |
| 0.40 | acrylates C10-C30 alkyl acrylate crosspolymer |
| 0.30 | imidazolidinylurea |
| 0.25 | hydroxyethylcellulose |
| 0.25 | sodium methylparaben |
| 0.20 | disodium EDTA |
| 0.15 | fragrance |
| 0.15 | sodium propylparaben |
| 0.10 | sodium hydroxide |

### Example 8

### Non-Greasy Gel

Mass content

(% by wt.)

| ad 100 | water |
|---|---|
| 8.00 | octyl methoxycinnamate |
| 7.00 | titanium dioxide, micronized |
| 5.00 | compound (c) in Table 1 |
| 5.00 | glycerol |
| 5.00 | PEG-25 PABA |
| 1.00 | 4-methylbenzylidenecamphor |
| 0.40 | acrylates C10-C30 alkyl acrylate crosspolymer |
| 0.30 | imidazolidinylurea |
| 0.25 | hydroxyethylcellulose |
| 0.25 | sodium methylparaben |
| 0.20 | disodium EDTA |
| 0.15 | fragrance |
| 0.15 | sodium propylparaben |
| 0.10 | sodium hydroxide |

### Example 9

### Sun Cream (SPF 20)

Mass content

(% by wt.)

| ad 100 | water |
|---|---|
| 8.00 | octyl methoxycinnamate |
| 8.00 | titanium dioxide, micronized |
| 6.00 | PEG-7 hydrogenated castor oil |
| 5.00 | compound (b) in Table 1 |
| 6.00 | mineral oil |
| 5.00 | zinc oxide |
| 5.00 | isopropyl palmitate |
| 0.30 | imidazolidinylurea |
| 3.00 | jojoba oil |
| 2.00 | PEG-45/dodecyl glycol copolymer |
| 1.00 | 4-methylbenzylidenecamphor |
| 0.60 | magnesium stearate |
| 0.50 | tocopheryl acetate |
| 0.25 | methylparaben |
| 0.20 | disodium EDTA |
| 0.15 | propylparaben |

### Example 10

### Sun Cream (SPF 20)

Mass content

(% by wt.)

| ad 100 | water |
|---|---|
| 8.00 | octyl methoxycinnamate |
| 8.00 | titanium dioxide, micronized |
| 6.00 | PEG-7 hydrogenated castor oil |
| 5.00 | compound (a) in Table 1 |
| 6.00 | mineral oil |
| 5.00 | zinc oxide |
| 5.00 | isopropyl palmitate |
| 0.30 | imidazolidinylurea |
| 3.00 | jojoba oil |
| 2.00 | PEG-45/dodecyl glycol copolymer |
| 1.00 | 4-methylbenzylidenecamphor |
| 0.60 | magnesium stearate |
| 0.50 | tocopheryl acetate |
| 0.25 | methylparaben |
| 0.20 | disodium EDTA |
| 0.15 | propylparaben |

### Example 11

### Sun Cream, Water-Resistant

Mass content

(% by wt.)

| ad 100 | water |
|---|---|
| 8.00 | octyl methoxycinnamate |
| 5.00 | PEG-7 hydrogenated castor oil |
| 5.00 | propylene glycol |
| 4.00 | isopropyl palmitate |
| 4.00 | caprylic/capric triglyceride |
| 5.00 | compound (b) in Table 1 |
| 4.00 | glycerol |
| 3.00 | jojoba oil |
| 2.00 | 4-methylbenzylidenecamphor |
| 2.00 | titanium dioxide, micronized |
| 1.50 | PEG-45/dodecyl glycol copolymer |
| 1.50 | dimethicone |
| 0.70 | magnesium sulfate |
| 0.50 | magnesium stearate |
| 0.15 | fragrance |

### Example 12

### Sun Cream, Water-Resistant

Mass content

(% by wt.)

| ad 100 | water |
|---|---|
| 8.00 | octyl methoxycinnamate |
| 5.00 | PEG-7 hydrogenated castor oil |
| 5.00 | propylene glycol |
| 4.00 | isopropyl palmitate |
| 4.00 | caprylic/capric triglyceride |
| 5.00 | compound (c) in Table 1 |
| 4.00 | glycerol |
| 3.00 | jojoba oil |
| 2.00 | 4-methylbenzylidenecamphor |
| 2.00 | titanium dioxide, micronized |
| 1.50 | PEG-45/dodecyl glycol copolymer |
| 1.50 | dimethicone |
| 0.70 | magnesium sulfate |
| 0.50 | magnesium stearate |
| 0.15 | fragrance |

### Example 13

### Sun Milk (SPF 6)

Mass content

(% by wt.)

| ad 100 | water |
|---|---|
| 10.00 | mineral oil |
| 6.00 | PEG-7 hydrogenated castor oil |
| 5.00 | isopropyl palmitate |
| 3.50 | octyl methoxycinnamate |
| 5.00 | compound (b) in Table 1 |
| 3.00 | caprylic/capric triglyceride |
| 3.00 | jojoba oil |
| 2.00 | PEG-45/dodecyl glycol copolymer |
| 0.70 | magnesium sulfate |
| 0.60 | magnesium stearate |
| 0.50 | tocopheryl acetate |
| 3.00 | glycerol |
| 0.25 | methylparaben |
| 0.15 | propylparaben |
| 0.05 | tocopherol |

### Example 14

### Sun Milk (SPF 6)

Mass content

(% by wt.)

| ad 100 | water |
|---|---|
| 10.00 | mineral oil |
| 6.00 | PEG-7 hydrogenated castor oil |
| 5.00 | isopropyl palmitate |
| 3.50 | octyl methoxycinnamate |
| 5.00 | compound (c) in Table 1 |
| 3.00 | caprylic/capric triglyceride |
| 3.00 | jojoba oil |
| 2.00 | PEG-45/dodecyl glycol copolymer |
| 0.70 | magnesium sulfate |
| 0.60 | magnesium stearate |
| 0.50 | tocopheryl acetate |
| 3.00 | glycerol |
| 0.25 | methylparaben |
| 0.15 | propylparaben |
| 0.05 | tocopherol |

1. A method of protecting skin or hair against solar rays, comprising, applying a composition comprising a diketopiperazine of the formula I, to the skin or hair, 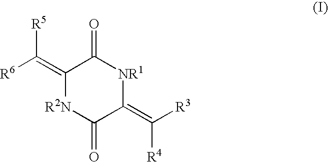
and wherein
R¹ and R² may, in each case, independently of one another, be identical or different, and are hydrogen or C₁-C₁₂-alkyl, and
R³, R⁴, R⁵, R⁶, may in each case, independently of one another, be identical or different, and are hydrogen, C₁-C₁₂-alkyl or an aryl radical,
with the proviso that at least one radical should be aryl, and
wherein the composition optionally comprises one or more additional compounds, which absorb in the UV region, known for cosmetic and pharmaceutical preparations.

2. A photostable UV-A filter, comprising the compound of formula I.

3. A method of stabilizing a cosmetic or pharmaceutical formulation, comprising, contacting the photostable UV-A filter, as claimed in claim 2, with one or more additives suitable for cosmetic or pharmaceutical formulations.

4. A composition, comprising an effective amount of a compound of the formula I, and at least one additive, and 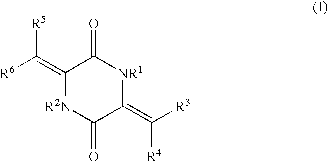
wherein
R¹ and R² may, in each case, independently of one another, be identical or different, and are hydrogen or C₁-C₁₂-alkyl, and
R³, R⁴, R⁵, R⁶ may, in each case, independently of one another, be identical or different, and are hydrogen, C₁-C₁₂-alkyl or an aryl radical,
with the proviso that at least one radical should be aryl, and
wherein the composition optionally comprises one or more additional compounds, which absorb in the UV region, known for cosmetic and pharmaceutical preparations.

5. The composition of claim 4, wherein the at least one additive comprises a cosmetically or pharmaceutically suitable carrier.

6. The method of claim 1, wherein protection is provided against UV light in a range from 280 to 400 nm.

7. The composition of claim 4, wherein the C₁-C₁₂-alkyl radical is a branched alkyl chain or unbranched alkyl chain.

8. The composition of claim 4, wherein the C₁-C₁₂-alkyl radical is a member selected from the group consisting of methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl and n-dodecyl.

9. The composition of claim 4, wherein the aryl radical is an aromatic ring or ring system, having 6 to 18 carbon atoms in the ring system.

10. The composition of claim 4, wherein the aryl radical is a member selected from the group consisting of benzyl, phenyl and naphthyl, and
wherein said benzyl, phenyl or naphthyl, each may optionally be substituted with one or more radicals.

11. The composition of claim 4, wherein the aryl radical is an optionally substituted phenyl, methoxyphenyl, cyanophenyl or naphthyl radical.

12. The method of claim 1, wherein the C₁-C₁₂-alkyl radical is a branched alkyl chain or unbranched alkyl chain.

13. The method of claim 1, wherein the C₁-C₁₂-alkyl radical is a member selected from the group consisting of methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, n-undecyl and n-dodecyl.

14. The method of claim 1, wherein the aryl radical is an aromatic ring or ring system, having 6 to 18 carbon atoms in the ring system.

15. The method of claim 1, wherein the aryl radical is a member selected from the group consisting of benzyl, phenyl and naphthyl, and
wherein said benzyl, phenyl or naphthyl, each may optionally be substituted with one or more radicals.

16. The method of claim 1, wherein the aryl radical is an optionally substituted phenyl, methoxyphenyl, cyanophenyl or naphthyl radical.
